(19) United States
(12) Patent Application Publication
(10) Pub. No.: US 2006/0194715 A1
(43) Pub. Date: Aug. 31, 2006

(54) Proteins encoded by ble genes and antibiotics from the bleomycin family

(76) Inventors: Jonathan Michael Blackburn, Cambridge (GB); Darren James Hart, Cedex (FR); Ben Godber, Maidenhead (GB); Mike McAndrews, Maidenhead (GB)

Correspondence Address: MINTZ LEVIN COHN FERRIS GLOVSKY & POPEO, 666 THIRD AVENUE, NEW YORK, NY, 10017, US

(21) Appl. No.: 10/532,834

(22) PCT Filed: Oct. 27, 2003
(86) PCT No.: PCT/IB03/05430

§ 371 Date: Feb. 16, 2006

(30) Foreign Application Priority Data
Oct. 25, 2002 (GB) 0224872.2
Dec. 20, 2002 (GB) 0229640.8
Mar. 31, 2003 (GB) 0307379.8

(51) Int. Cl. G01N 33/554; C07K 9/00; A61K 38/14

(52) U.S. Cl. 514/8; 530/322; 435/7.32

(57) ABSTRACT

The present invention provides use of a protein conjugate comprising a “ble” protein, which has specific binding properties. The protein conjugates are capable of binding reversibly to an antibiotic from the bleomycin family, which property is exploited in a variety of immobilisation methods. In preferred aspects of the invention, the conjugates are used as markers for protein expression and/or folding, or for affinity tagging. The present invention also provides a probe comprising an array of an immobilised antibody from the bleomycin family, which acts as an analyte capture moiety. In another aspect, a purification media is provided, which comprises an antibiotic of the bleomycin family as an analyte capture moiety. Also provided is a method for generating soluble forms of an insoluble protein by expressing the protein as a “ble” fusion protein and selecting in the presence of an antibiotic from the bleomycin family. In a further aspect, the “ble” protein is expressed as a fusion protein in a cell into which is introduced a labelled antibiotic of the bleomycin family, thereby allowing identification of the cellular localisation of the protein.

### CROSS REFERENCE TO RELATED APPLICATIONS

This application claims priority to GB0307379.8, filed Mar. 31, 2003, GB0224872.2, filed Oct. 25, 2002 and GB0229640.8, filed Dec. 20, 2002 each of which is hereby incorporated by reference in its entirety.

### FIELD OF THE INVENTION

The present invention relates to new uses for the family of “ble” genes, for example Sh ble, Tn5 ble and Sa ble, and the family of proteins expressed by these genes which are able to reversibly bind to the bleomycin family of antibiotics. The bleomycin family of antibiotics are DNA—cleaving glycopeptides and include bleomycin, phleomycin, tallysomycin, pepleomycin and Zeocin™. The invention also relates to new uses of these antibiotics. More particularly it relates to fusion proteins comprising a ble protein and tools, methods and products which depend on the specificity between the ble protein and the antibiotics of the bleomycin family.

When these ble genes are expressed together with another protein as fusion proteins or the ble gene products are otherwise fused or linked to other molecules, particularly proteins, they can, for example, be used to strongly or weakly bind the fusion protein to a surface, such as an array, particular a microarray, a glass slide or a microtitre plate (where strong binding is generally desired) as well as to, for example, beads or other similar forms which are used in affinity purification (where weak binding is generally desired).

The invention however relates not only to the use of this pairing in binding molecules to surfaces, particularly though not essentially arrayable surfaces, but also to the use of a ble fusion protein as a folding and solubility marker. It also relates to the use of “labelled” antibiotics from the bleomycin family which due to their specificity to the ble proteins make them useful as markers of cellular localisation.

### BACKGROUND OF THE INVENTION

Expression of human proteins in heterologous systems such as bacteria, yeast, insect cells or mammalian cells can result in the production of incorrectly folded proteins resulting in the formation of insoluble aggregates and/or a low yield of expressed proteins. For all functional proteomic work the production of correctly folded or native proteins is essential and a great deal of work is often performed to optimise the expression of individual proteins. Two areas where incorrect folding and poor solubility may be significant are set out below:

Problematic Insolubility of Engineered Proteins

The manipulation of protein function by genetic engineering is a major field of study with strong commercial and academic drivers. The desired characteristics sought from such engineered proteins include altered substrate specificity, novel catalytic function, improved binding specificity and improved thermal or pH tolerance. However, the primary mutations introduced into the protein frequently result in loss of soluble expression and compensating mutations to restore solubility are usually impossible to predict.

Problematic Insolubility of Proteins Expressed Recombinantly in Heterologous Hosts

Expressing recombinant proteins in Escherichia coli is often preferred, especially for bulk protein production (e.g. industrial enzymes, for X-ray crystallography), due to the ease of fermentation, absence of heterogeneous post-translational modification, simplicity of subsequent processing steps and high yields of material. However a frequent problem encountered, especially with eukaryotic proteins, is that the recombinant material is expressed insolubly and is therefore inactive. Such problematic proteins may be expressed solubly in more complicated systems (e.g. Pichia pastoris, baculovirus), but the lower yields and more expensive process can result in a commercially less attractive product.

In both of these cases, a method for generating soluble forms of an insoluble protein has great utility. Random mutagenesis of the protein to generate a library of variants followed by selection or screening for soluble expression is one approach. This is often referred to as “directed evolution”. The requirements of such a screen are that many variant clones can be assessed in parallel since the libraries involved are large (often >10⁶ members). In some cases the activity of the target protein may be assayed directly. However, this is relatively rare and often requires each clone to be lysed in the wells of a microtitre plate and often the proteins have no assayable activity.

A more efficient method is to fuse to the protein a second “reporter” protein with a visually assayable phenotype. Since the two proteins are physically linked, the solubility of the reporter (and therefore presence of the observable phenotype) is dependent on the solubility of the other. Several such reporter systems have been reported. Preferably the phenotype can be observed with no process steps (i.e. when clones are still growing as colonies on agar plates) permitting a high throughput analysis.

Large scale expression of recombinant proteins for the manufacture of protein arrays is one area of proteomics which involves the functional characterisation of proteins in a parallel manner. Many thousands of proteins are required to be expressed in a soluble and folded manner. Expression libraries are usually employed whereby genes are placed under the control of a promoter and expression of the gene is then induced. The next step is to assess which clones express folded recombinant protein. On an individual basis, this is usually achieved by fractionating cell lysates into soluble and insoluble components by centrifugation and subsequent analysis of the fractions by gel electrophoresis. This approach is very low throughput and scale up to the level required to screen libraries is logistically infeasible. Thus, a need exists for a method to screen expression libraries for clones that produce soluble, folded proteins that may then be used for functional studies. Fusion of the recombinant proteins to a reporter protein permits simple determination of its solubility.

Before considering what has been done to date it is necessary to make the important distinction between a “screen” in which all members of a collection are assessed with a subset conforming to the desired characteristics being isolated, and a “selection” in which only the members of interest are observable.

Selections are more powerful than screens when dealing with very large numbers since practical limitations exists associated with handling and analysing large numbers of clones. Common examples of screens used in molecular biology are blue/white selection and GFP fluorescence.

Solubility/folding reporters resulting in a colour based (visual) screen that have been described use β-galactosidase (Wigley, W. C., Stidham, R. D., Smith, N. M., Hunt, J. F. & Thomas, P. J. Protein solubility and folding monitored in vivo by structural complementation of a genetic marker protein. Nat. Biotechnol. 19, 131-135 (2001); and green fluorescent protein (GFP). (Waldo, G. S., Standish, B. M., Berendzen, J. & Terwilliger, T. C. Rapid protein-folding assay using green fluorescent protein Nat. Biotechnol. 17, 691-695 (1999).) Optimisation of protein solubility by directed evolution for use in structural studies has also been performed using GFP. (Pédelacq, J. D. et al. Engineering soluble proteins for structural genomics. Nat. Bioteclnol. 20, 927-932 (2002).)

Several examples of exploiting fusion markers as indicators of protein folding and solubility exist in the literature. The principle that underlies these systems is the observation that protein folding and solubility are closely correlated since misfolded protein usually forms insoluble aggregates or are heavily proteolysed by the host cell. It is therefore assumed that if a protein is solubly expressed in an unproteolysed form, it is in its correctly folded form. If a fusion is made between one protein and another, the folding and solubility of one domain is linked to that of the other. This is shown in FIG. 1 which illustrates the principle of a folding marker for assessing solubility of the gene X expression product. Only when the protein product of gene X is soluble is the phenotype of the fusion apparent. In this case, the fusion results in green fluorescent colonies that can be clearly identified.

A life-or-death selection for solubility and folding has been described employing chloramphenicol acetyl transferase (CAT) (Maxwell, K. L., Mittermaier, A. K., Forman-Kay, J. D. & Davidson, A. R. A simple in vivo assay for increased protein solubility. Protein Sci. 8, 1908-1911 (1999).)

Definitions

As defined herein “ble” genes are a family of genes which express proteins which reversibly bind the glycopeptide antibiotics of the bleomycin family, and include, but are not limited to Sh ble, Tn5 ble and Sa ble. In general these genes (or more precisely the gene products encoded by these genes) confer to their host resistance to the glycopeptide antibiotics of the bleomycin family.

The “bleomycin family of antibiotics” are DNA—cleaving glycopeptides and include, but are not limited to, bleomycin, phleomycin, tallysomycin, pepleomycin and Zeocin™.

The term “proteins”, as used herein, is used to include both whole proteins, polypeptides, and sub units or domains thereof.

A “fusion protein”, as used herein, refers to a protein, which comprises a “tag” at the N and/or C terminus which binds to members of the bleomycin family of antibiotics. The fusion protein may be expressed as a fusion protein from a ble gene containing genetic construct or may be formed by either intein mediated splicing of, for example, two separate polypeptides or by the chemical ligation of two such peptides according to methods known in the art.

The “tag” is then used as a signalling component and/or a means to manipulate the fusion protein further. Thus, it may be used to bind the fusion protein to a surface or to another molecule, such as for example a visual indicator molecule, such as a fluorescent molecule, or to indicate a characteristic of the fusion protein e.g. that it is folded such that it is likely to be functional.

The relative terms “strong binder” and “weak binder” and “high density” and “low density” are used herein to functionally distinguish between examples where it is desirable to achieve a substantially irreversible bond and the situation where it is intended that binding will be reversed such as in affinity purification. Thus, because for example the Sh ble protein is a dimer that can bind two molecules of bleomycin co-operatively (the first affinity is ˜600 nM whilst the second affinity is ˜100 nM) it is possible to use the ble proteins binding characteristics in a selective manner. For example, if one has a surface (e.g. a bead) coated with bleomycin at low density, then one would expect on average that one Sh ble dimer would be bound by one bleomycin molecule. In contrast, if one has a surface (e.g. a bead) coated with bleomycin at a high density, then one would expect on average that one Sh ble dimer would be bound by two bleomycin molecules (each binding one of the two dimer subunits), particularly if the bleomycin molecules were themselves attached to flexible linkers (e.g. polyethyleneglycol (PEG) polymers). Since in this latter case the two bleomycin molecules are effectively linked together via the surface, the second binding affinity will now be several orders of magnitude greater than where the two molecules are not linked together. In other words, the binding affinity will be significantly enhanced by a chelate effect. This effect can be utilised to advantage since one would want different binding affinities in, for example, an array (where one wants a substantially irreversible binding) and a reversible capture system such as a bead.

So, by controlling the surface density of the antibiotic, such as, bleomycin, it should be possible to readily control whether the Sh ble fusions bind weakly or strongly to the surface. Weak binding would be appropriate for affinity purifications whilst strong binding would be appropriate for immobilisation into arrays.

As an alternative to immobilising the bleomycin molecules onto a surface, one can covalently couple them to, for example amine-reactive fluorescent dyes, such as NHS-activated fluorescein, Cy3, Cy5, and Rhodamine. It should then be possible to diffuse the fluorescent bleomycin derivatives into whole cells (both prokaryotic and eukaryotic cells), whereupon the derivatives would bind selectively to Sh ble fusion proteins. In this way the Sh ble tag can be used as a marker of cellular localisation (in the same way as GFP is currently used). This will also enable the Sh ble tag to be used as both a selectable (i.e. life-or-death) marker and a screenable (i.e. fluorescence-based) marker.

In one aspect of the invention the support takes the form of a probe which is capable of acting as a target in analysis by laser desorption/ionisation mass spectrometry, for example, matrix assisted laser desorption/ionisation (MALDI). The probe carries the analytes, for example proteins, during such processes and interacts with the repeller lens of the ion-optic assembly found in laser desorption/ionisation time-of-flight (TOF) mass spectrometers of the art, such that the analytes are converted to gaseous ions to permit analysis. For example, the probes of the invention may be derived from targets for MALDI analysis as known in the art, which are treated such that a high affinity protein binding moiety e.g. Zeocin are present on the probe surface which bind Sh ble fusion proteins for subsequent analysis. For example, conventional glass or gold MALDI targets may be used.

As defined herein a “micro array” is an array where the size of the discrete target areas i.e. the individual areas probed by a laser, is in the order of micrometers or less. Whilst at the upper end of the scale, around 1000 micrometers diameter, they may be visible to the naked eye, at the lower end of the scale the discrete target areas will not be clearly distinguished by the naked eye.

The “arrays” will typically be arranged in matrices comprising several rows and columns. The number of discrete target areas will depend upon what is being screened though it is generally desirable to have a high density of these discrete areas on the probe surface as this will facilitate high through put screening. Typically a probe will comprise at least 10, more preferably at least 100, more preferably at least 1000 and as many as 10,000 or more target areas produced thereon. (Typically a probe surface will have an area of around 10,000 mm²—a Bruker probe has an area of 10292 mm² although there is no requirement to use the whole of the probe and the microarray can be applied in one or more matrices thereon.) The actual density in a given matrices will depend upon the size of the discrete target area (which will typically be printed as a spot) and the spacing between adjacent spots. Thus the discrete target areas will typically be present at a density of greater than 1 discrete target areas per mm² within any matrices.

“Linker molecules” are molecules which function as their name suggests. They are molecules comprising functional groups which allow bridges to be formed between different molecules.

### SUMMARY OF THE INVENTION

The protein product of, for example, the Sh ble gene encodes a protein that when expressed by a cell, confers resistance to antibiotics of the bleomycin family including Zeocin by binding to the antibiotic and sequestering it. (Gatignol, A., Durand, H. & Tiraby, G. Bleomycin resistance conferred by a drug-binding protein. FEBS Lett 230, 171-175. (1988)).

These antibiotics otherwise induce DNA strand breakage. When, for example, the Sh ble gene is fused in tandem with a second gene of interest, thereby encoding a fusion protein, expression of the fusion protein also confers resistance to Zeocin. If the gene of interest encodes an insoluble protein or protein fragment, the Zeocin resistance-conferring phenotype is not observed since the fusion protein is insoluble and incapable of binding the antibiotic. This forms the basis of a novel life-or-death selection for folded, soluble proteins.

Additionally, since, for example, the Sh ble protein binds Zeocin tightly and co-operatively the inventors have shown that it can be used as an affinity tag if a surface is provided that is derivatised with, for example, Zeocin or an analogue.

Furthermore, by, for example, linking a visual indicator to the antibiotic and allowing the labelled antibiotic to bind the fusion protein via the tag it should be possible to use the ble fusion protein as, for example, a marker for cellular localisation.

According to a first aspect of the present invention there is provided the use of a ble fusion protein as an expression and folding marker and/or an affinity tag.

In one embodiment the ble fusion protein is used solely as a folding marker.

In another embodiment the ble fusion protein is used solely as an affinity tag.

In a preferred embodiment the ble fusion protein is used as both a folding marker and an affinity tag.

According to a second and related aspect of the present invention there is provided a method of immobilising a protein to a surface wherein the protein is provided to the surface as a ble fusion protein and the surface is a surface derivatised with an antibiotic from the bleomycin family.

Antibiotics from the bleomycin family include, but are not limited to bleomycin, phleomycin, tallysomycin, pepleomycin and Zeocin™. Preferred members include

bleomycin A2, bleomycin A5, bleomycin A6, bleomycin B2 and Zeocin™.

An advantage with bleomycin A5 and A6 is that they comprise a terminal primary amine group which can be used to couple the protein to, for example, an amine reactive surface. Typical groups used to produce an amine reactive surface include, but are not limited to: aldehydes, epoxides, N-hydroxysuccinamides, and isothionates. Of course other functional groups present on the antibiotics may be used to couple the antibiotics to a surface, and this will be apparent to the person skilled in the art.

In a preferred example coupling may be achieved by reacting the amine group with an amine reactive surface, such as, for example, a NHS activated, polyethyleneglycol (PEG) derivitized surface.

In a preferred embodiment the surface is the surface of an array, more particularly a microarray, and more particularly still a MALDI array although it could be a microtitre plate, a glass slide, (the surface being the surface of a probe) or a bead, (the surface being that of a purification media).

Thus according to a third aspect of the present invention there is provided a probe characterised in that it has a target surface comprising an array having a plurality of discrete target areas presenting one or more analyte capture moieties comprising an antibiotic from the bleomycin family.

Such a target surface typically has a high density coating of the antibiotic such that the target analyte is captured with a high affinity, preferably less than 400 nM, more preferably less than 200 nM, more particularly still less 150 nM. In the specific example noted the Sh ble has an affinity in the order of 100 nM or less when two molecules of bleomycin are bound.

According to a fourth aspect of the present invention there is provided a purification media comprising a target surface presenting one or more analyte capture moieties comprising an antibiotic from the bleomycin family.

Such a target surface typically has a low density coating of the antibiotic such that the target analyte is captured with a low affinity, typically in the order of greater than 400 nM, and more preferably greater than 500 nM. In the specific example noted the Sh ble has an affinity in the order of 600 nM when a single molecule of bleomycin is bound. Ideally the affinity would be in the μM range but sufficient that the proteins are bound.

In either case the antibiotic may be linked to the surface via a flexible linker such as an activated polyethyleneglycol (PEG).

According to a fifth aspect of the present invention there is provided an antibiotic from the bleomycin family characterised in that it is tagged with a marker.

Preferably the marker is a visual marker such as, for example, a fluorescent marker. Alternatively it could be labelled with a radioactive isotope. Preferred fluorescent markers include, but are not limited to NHS activated fluorescein, Cy3, Cy5 and Rhodamine.

A tool for the production of the fusion proteins is a vector consisting essentially of a ble gene downstream of a linker DNA which in use is excised and replaced with a DNA encoding a protein to be expressed as a ble fusion protein.

In one embodiment the ble vector is provided as one of the components of a kit for making an array along with a surface which has been derivatised with an antibiotic from the bleomycin family or the components for making such a derivatised surface.

According to a sixth aspect of the present invention there is provided a method for generating soluble forms of an insoluble protein comprising:
i) generating a library of protein variants; and
ii) selecting colonies for the presence of a soluble protein by expressing the protein as a ble fusion protein and selecting on an antibiotic from the bleomycin family.

Preferably the method further comprises growing up the selected colonies in liquid culture, lysing and printing a crude lysate onto a surface, and washing the tagged surface to remove non-bound materials.

A previous life-or-death selection utilising chloramphenicol acetyl transferase (CAT) is known, but is not very effective (Maxwell, K. L., Mittermaier, A. K., Forman-Kay, J. D. & Davidson, A. R. A simple in vivo assay for increased protein solubility. Protein Sci. 8, 1908-1911 (1999).) due to its narrow window of toxicity. i.e. the concentration of antibiotic that kills cells with no CAT is close to that which permits survival of cells with CAT. Also, it does not directly enable the subsequent downstream purification or immobilisation of the expressed protein since CAT enzymatically destroys the antibiotic and does not bind the product with significant affinity.

According to an seventh aspect of the present invention there is provided a method of purifying a Sh ble fusion protein from a crude extract comprising the step of immobilising it on a surface via an antibiotic from the bleomycin family and optionally releasing it there from.

According to a eighth aspect of the invention there is provided a method of identifying the cellular localisation of a protein comprising

i) expressing the protein as a ble fusion protein in a cell,

ii) introducing a labelled antibiotic from the bleomycin family into the cell, and detecting the labelled antibiotic.

The method may be qualitive or quantitive in which case the method comprises a further step of quantitating the labelled antibiotic. This may be done by measuring the intensity of fluorescence where the labelled antibiotic is fluorescently labelled.

### BRIEF DESCRIPTION OF THE DRAWINGS

The various aspects of the invention are further described by way of example only with reference to the following Figs and Examples in which:

FIG. 1 is a schematic demonstrating the principle of using a folding marker for assessing the solubility of a “gene X” expression product. Only when the protein product of gene X is soluble is the phenotype of the fusion apparent. In this case, the fusion results in green fluorescent colonies that can be clearly identified;

FIG. 2 is a vector according to one aspect of the invention incorporating a Sh ble gene;

FIGS. 3a and b illustrate the purification of a Sh ble fusion protein on Bleomycin Sepharose

A) is a Coomassie stained gel, and

B) is a Streptavidin HRP probed western.

Lanes 1-6 are loaded as follows:

1) lysate,

2) Bleomycin-Sepharose after incubation with lysate,

3) unbound lysate after incubation with Bleomycin-Sepharose,

4) Bleomycin elution,

5) Bleomycin-Sepharose after Bleomycin elution, and

6) unbound lysate after incubation with Bleomycin-Sepharose.

FIG. 4 illustrates the immobilisation of Sh Ble fusion proteins on a Bleomycin derivatised microtitre plate. Lysates of two different Sh ble fusion protein clones were added to wells of Bleomycin derivatised and control microtitre plate in the presence and absence of competing Bleomycin;

FIG. 5 illustrates the purification of immobilised Sh ble fusion proteins on Bleomycin coated microwells; (Sh ble fusion protein captured from lysates on Bleomycin derivatised microwells was run on SDS PAGE. Input lysates were also run for comparison. Input lysates were a 2 fold dilution series, with lane 1 representing undiluted lysate and lane 7 representing a 1 in 64 dilution.)

FIG. 6 shows the rate of immobilization of Sh ble fusion protein on Bleomycin derivatised microwell; (Cleared lysates of Sh ble fusion protein clone 1 were incubated for varying times in a Bleomycin derivatised microwell. A) Silver stained SDS PAGE showing amount of fusion protein immobilized. B) The bands were quantified and plotted against time.)

FIG. 7 illustrates the retention of Sh ble fusion protein on Bleomycin derivatised microwell; (Sh Ble fusion protein clone 1 was bound to Bleomycin derivatised microwells. After washing the wells were incubated for varying times in buffer.

A) Coomassie stained SDS PAGE showing amount of fusion protein remaining immobilized

B) The bands were quantified and plotted against time.)

FIG. 8 illustrates retention of Sh ble fusion protein on Bleomycin derivatised microwell. (Sh ble fusion protein clone 1 was bound to Bleomycin derivatised microwells. After washing the wells were incubated for varying times in buffer containing excess Bleomycin. A) Coomassie stained SDS PAGE showing amount of fusion protein remaining immobilized. B) The bands were quantified and plotted against time.

and

FIG. 9 illustrates immobilisation of Sh ble fusion protein on a Bleomycin derivatised microarray slide. (1 μl of cleared lysates of Sh ble fusion protein clone 2 were spotted onto a bleomycin derivatised micro-array slide in the presence and absence of excess free bleomycin. A) After probing for the fusion protein using an anti Flag antibody and Cy3 labelled anti mouse secondary the Cy3 fluorophore was visualized. B) Total spot fluorescence intensity was plotted in a histogram for spots in the presence and absence of excess Bleomycin.)

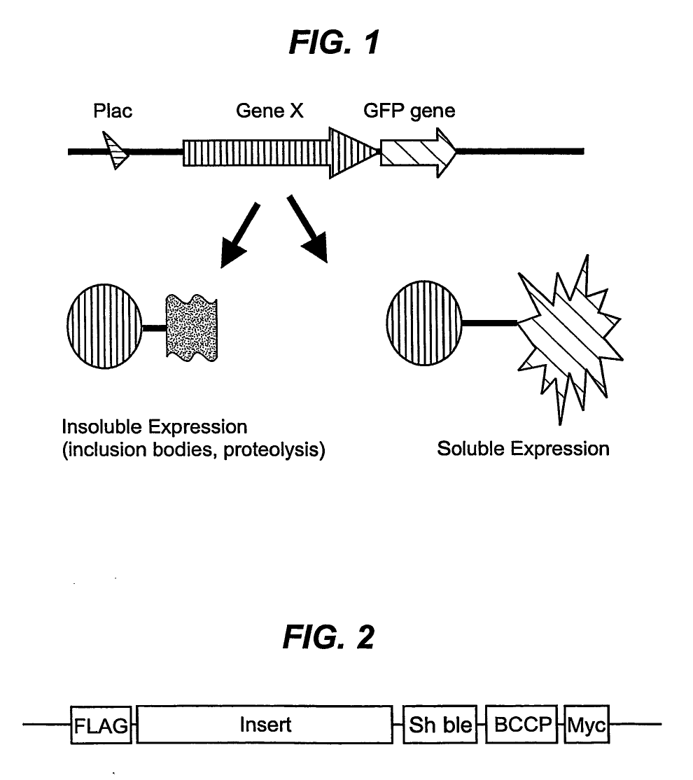

### DETAILED DESCRIPTION OF THE INVENTION

1.0 Use of Sh ble as an Affinity Tag

A cDNA library was ligated into a modified E. coli expression vector (compare to FIG. 2) so that the gene could be Tagged at both the N and C terminii with a FLAG Tag at the N terminus and Sh ble, BCCP (Biotinylated domain of E. coli ACCB) and Myc Tag at the C terminus.

After transformation into XL10 Gold, clones were selected by growing on LB Agar containing 50 μg/ml Zeocin.

Two Zeocin resistant clones were selected, grown in LB-amp to an OD₆₀₀ of approx 0.4 after which time the clones were induced with 100 μg/ml IPTG for 4 h at 30° C. The cells were harvested by centrifugation, 4000 g for 15 min at 4° C., and stored in aliquots at −20° C. to be used for future work. These are referred to as “Clone 1” and “Clone 2”.

Fresh E. coli lysates were prepared prior to each experiment.

To each aliquot of cells (from 40 ml of induction culture) 300 μl of lysis buffer (200 mM Tris pH 8, 100 mM EDTA, 1× Protease inhibitor cocktail (Roche), 10 mM mercaptoethanol, 200 μg/ml lysozyme) was added and the cells resuspended. After 15 min incubation at 4° C. cells were diluted with 1.7 ml of buffer (1× protease inhibitors, 20 mM MgCl₂, 50 μg/ml DNAse) and incubated at 4° C. for a further 30 minutes. Cleared lysates were obtained after centrifugation 20000 g for 10 min at 4° C.

1.1 Derivatisation of Sepharose with Bleomycin and its use in Affinity Purification.

Bleomycin A6 (2 mg/ml) was coupled to a Pharmacia 1 ml HiTrap NHS activated Sepharose column according to the manufacturers instruction.

After coupling the Sepharose was removed from the column and used for batchwise purification of Sh ble fusion proteins.

To 6 μl of Bleomycin-Sepharose slurry (approx 50% swollen beads) in PBS-Tween 4 μl of Clone 1 lysate (prepared as above) was added and incubated at 4° C. for 30 min with shaking. After briefly centrifuging the supernatant was removed and the beads were washed three times in 10 μl of PBS-Tween. To a similarly treated batch of Bleomycin-Sepharose bound protein was eluted by incubating the beads in 10 μl of PBS-Tween containing 300 μg/ml Bleomycin A6 at 4° C. for 10 min with shaking.

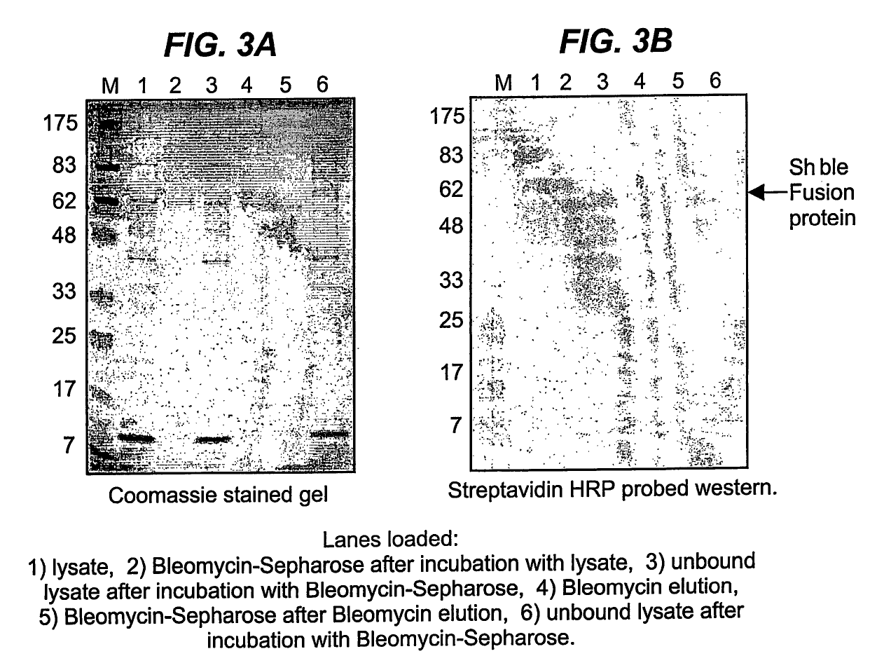

Samples were then boiled in SDS sample buffer and loaded onto two identical 10-20% Tris Glycine SDS PAGE plates. After electrophoresis one gel was stained with coomassie and the other transferred to nitrocellulose membrane and probed with streptavidin HRP before visualization of bands using DAB (FIG. 3).

Referring to FIG. 3 it can be clearly seen that the Sh ble fusion protein has bound to the Bleomycin sepharose on both the coomassie stained gel and Western Blot (FIG. 3 A and B lanes 2). It can also be seen that the Sh ble fusion protein bound is very pure producing only one band on Coomassie stained gel. The protein has not only shown to be specifically bound and significantly depleted from the lysate of fusion protein, but also can be specifically eluted with free bleomycin (FIG. 3 A and B lanes 4) thus providing evidence of both specificity of interaction and also a means of elution. However under these conditions only a proportion of the Sh ble fusion protein was eluted from the Bleomycin-Sepharose.

1.2 Derivatisation of Microtitre plates with Bleomycin

Microtitre 8 well strips coated with PEG with an amine reactive terminus (“protein immobilizer” plates) were purchased from Exiqon. Plates were coated with Bleomycin A6 effectively according to the manufacturers protocol. Briefly Bleomycin A6 was dissolved in 100 mM KPO₄ buffer pH 8 at a concentration of 1 mg/ml. 100 μl of Bleomycin solution was added to each well, the wells were sealed and incubated at 4° C. for 18 h. After which the Bleomycin solution was removed from the wells and the wells were washed with PBS-Tween for 5 minutes three times. Control wells were prepared as above with the omission of bleomycin A6.

1.3 Use of Bleomycin Surfaces in Affinity Immobilization

To Bleomycin coated and control wells, 50 μl of two different Sh ble fusion protein clone lysates (Clone 1 and Clone 2 lysates prepared as above) were added in the presence and absence of 5 mg/ml bleomycin. Lysates were incubated in the wells for 30 mins at room temperature with slight agitation, after which time the lysate was aspirated from the wells and the wells were washed three times with PBS-Tween for 5 minutes each wash.

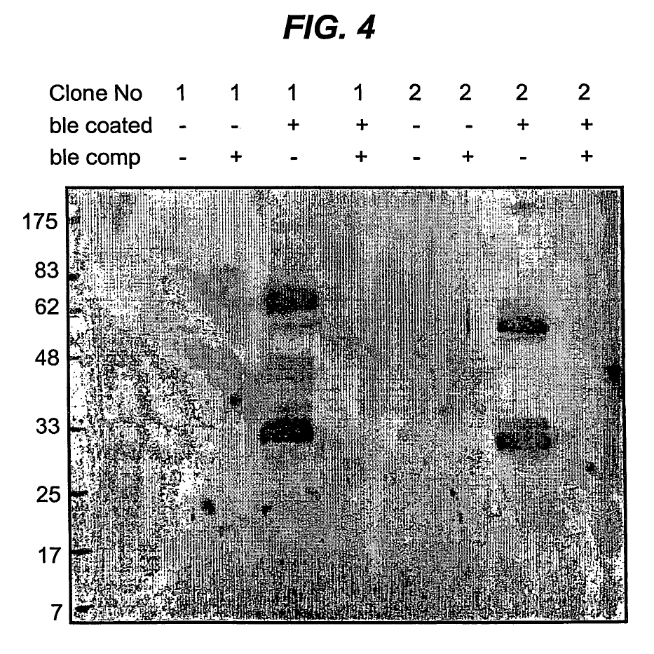

Proteins were eluted from the surface by the addition of 50 μl of SDS sample loading buffer and the wells were heated on a heating block at 100° C. for 10 mins. 20 μl of each sample was run on 10-20% Tris Glycine SDS PAGE. The gel was transferred to nitrocellulose membrane and probed using streptavidin HRP conjugate. The Blot was visualized using ECL (Amersham) and photographic film shown in FIG. 4.

From FIG. 4 it is clear that both Sh ble fusion proteins are only immobilized on Bleomycin derivatised micro wells and in addition that this interaction can be competed by pre-incubation of lysates with excess free bleomycin.

1.4 Analysis of Sh ble Fusion Protein Purified and Immobilized on Bleomycin Derivatised Microwells

50 μl of Cleared lysates at differing dilutions of Sh ble fusion Clone 1 prepared as above were added to Bleomycin derivatised microwells. After incubation for 40 minutes at room temperature the wells were washed three times with PBS-Tween. Protein was eluted from the wells by heating on a heating block at 100° C. for 10 min in SDS sample buffer and 15 μl were electrophoresed on SDS PAGE with equivalent amounts of input lysates. After electrophoresis gels were visualised using a standard silver staining protocol.

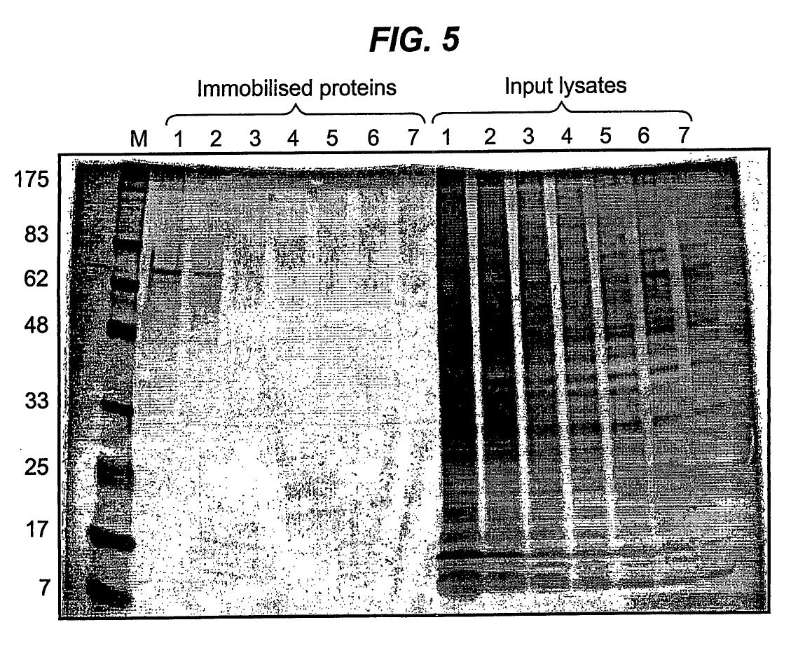

The results are illustrated in FIG. 5 which shows purification of immobilised Sh ble fusion proteins on Bleomycin coated microwells. Sh ble fusion protein captured from lysates on Bleomycin derivatised microwells was run on SDS PAGE. Input lysates were also run for comparison. Input lysates were a 2 fold dilution series, with lane 1 representing undiluted lysate and lane 7 representing a 64 fold dilution.

FIG. 5 demonstrates that there is predominantly 1 major band of purified full length Sh Ble fusion protein captured on microwells. It is probable that other minor bands seen represent proteolysis products of the Sh ble fusion as shown by comparison to a similar experiment with western blotting shown in FIG. 4.

1.5 Rate of Binding of Sh ble Fusion Protein to Bleomycin Derivatised Microwells

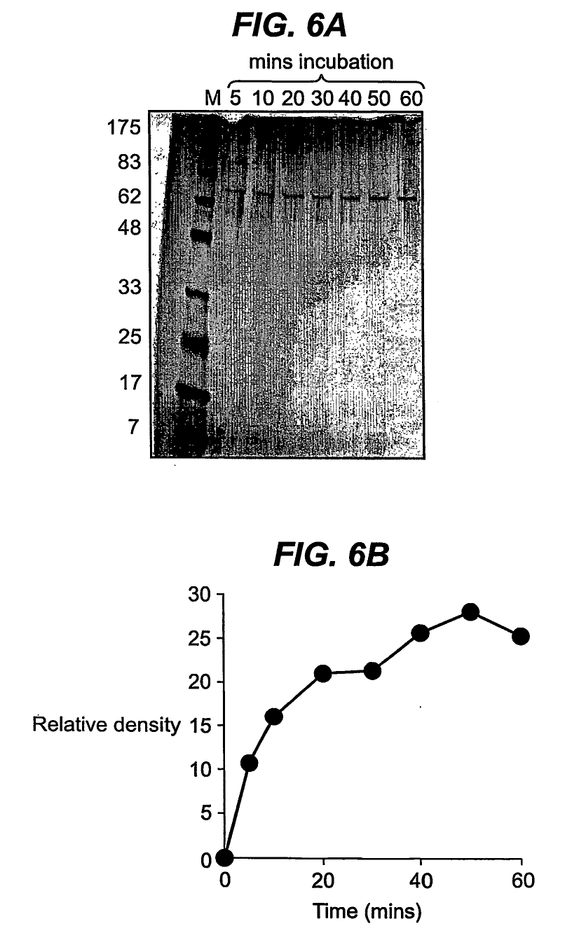

50 μl of cleared lysate of Sh ble fusion Clone 1 were added to Bleomycin derivatised microwells. After 5, 10, 20, 30, 40, 50 and 60 minutes of incubation at room temperature the wells were washed three times with PBS-Tween. Protein was eluted from the wells by heating on a heating block at 100° C. for 10 min in SDS sample buffer and 15 μl were electrophoresed on SDS PAGE. After electrophoresis gels were visualised using a standard silver staining protocol (FIG. 6 A). The Sh ble bands on the gel image were quantified and average density was plotted (FIG. 6 B). These data show that most protein is immobilized after approximately 40 minutes of incubation.

1.6 Stability of Sh ble Fusion Protein Interaction with Bleomycin Derivatised Microwells.

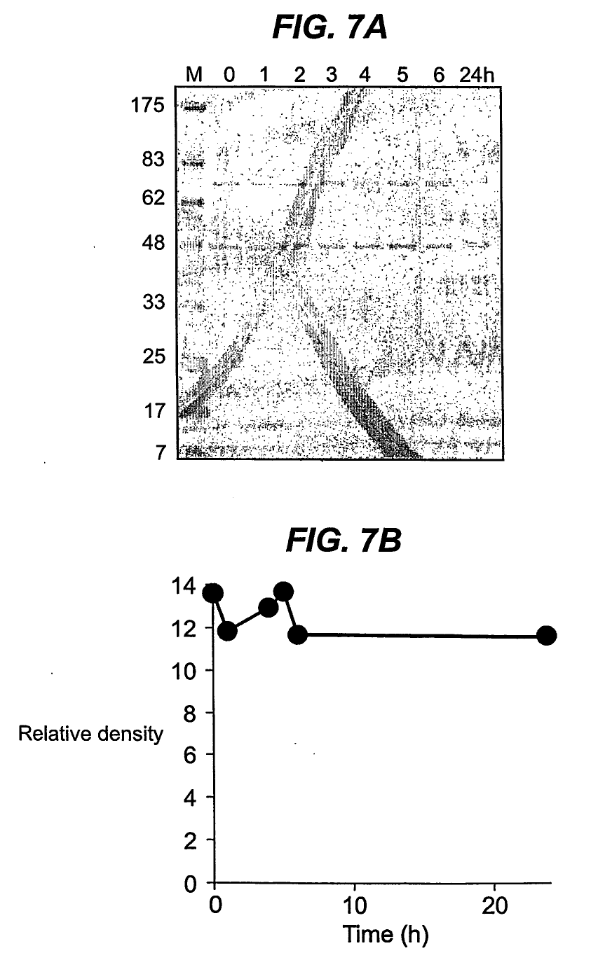

50 μl of cleared lysate of Sh Ble fusion Clone 1 were added to Bleomycin derivatised microwells. After incubation for 1 h at room temperature the wells were washed three times with PBS-Tween. After washing wells were filled with PBS-Tween and incubated for the desired time to generate a time course. This was also repeated with a duplicate set of wells, which after washing the wells were incubated in 0.5 mM Bleomycin in PBS-Tween. After these time courses, protein was eluted from the wells by heating on a heating block at 100° C. for 10 min in SDS sample buffer and 15 μl were electrophoresed on SDS PAGE with equivalent amounts of input lysates. After electrophoresis gels were visualised using coomassie stain (FIGS. 7A & 8A).

The Sh ble fusion protein bands on the gel image were quantified and average density was plotted (FIGS. 7B & 8B).

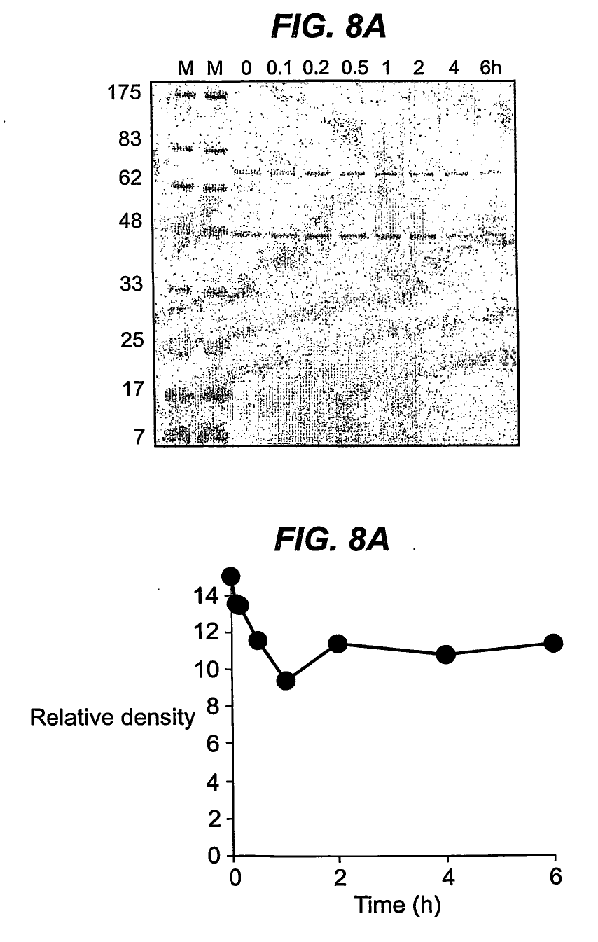

Immobilised Sh ble fusion protein remained very largely bound to the Bleomycin derivatised microwell after incubation for up to 24 h in PBS-Tween (FIG. 7). The inclusion of excess free Bleomycin in the buffer was anticipated to increase the rate of fusion protein dissociation from the microwell, however even in the presence of excess free Bleomycin (FIG. 8) little loss of immobilized Sh ble fusion protein was observed.

1.7 Use of Sh ble Fusion for Immobilization of Proteins on Microarrays.

Exiqon Euray slides were used in this study and have a surface coating that is essentially that found in the microtitre plate wells used previously, where a PEG molecule with an amine reactive terminus is used to coat the slide. Derivatisation of these slides was performed as for the microtitre plate wells with 400 μl of 3 mg/ml Bleomycin A6 applied to the surface under a microscope slide hybridization chamber.

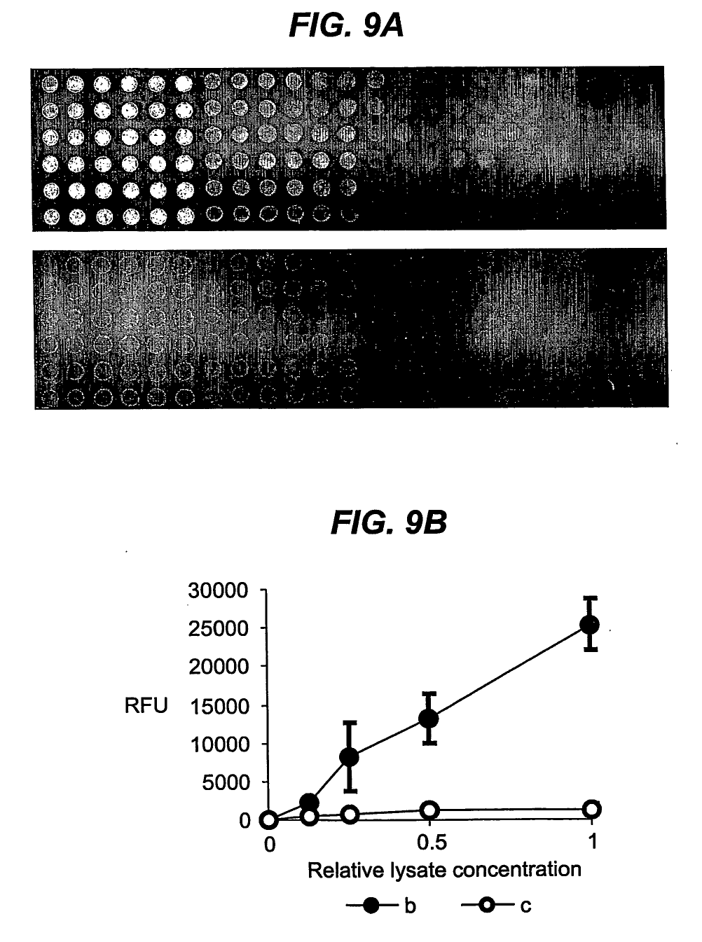

Cleared lysates of Sh ble fusion protein clone 1 were diluted 1 in 2, 1 in 4 and 1 in 8 with 100 mM KPO₄ buffer. The lysates were transferred to a 384 well microtitre plate for microarraying. A six by six pattern of the lysates was microarrayed onto one slide derivatised with bleomycin and one that had been similarly coated with ethanolamine. Microarraying was achieved using a Genetix Qarray robot equipped with 300 micron solid pins. The microarrays were incubated in a humid chamber for 1 h at room temperature. After washing in PBS-Tween the slides were probed with a mouse anti FLAG antibody followed by a Cy3 labelled anti mouse IgG antibody. After drying in a stream of nitrogen, slides were visualized using an Affymetrix 428 array scanner equipped with laser and filter appropriate for detecting the Cy3 Fluorophore (FIG. 9A). The total spot fluorescence was quantified and averages of the identical spots were taken and plotted (FIG. 9B).

The data in FIG. 9 demonstrate that a Sh ble fusion protein is immobilized specifically on a bleomycin derivatised microscope slide. This indicates that Sh ble is suitable for use as an affinity tag for the immobilization of proteins in a microarray format.

1-39. (canceled)

40. A Ble fusion protein wherein said protein is an expression and folding marker and/or an affinity tag.

41. The protein of claim 40, wherein said protein is an expression and folding marker.

42. The protein of claim 40, wherein said protein is an affinity tag.

43. The protein of claim 40, wherein said protein an expression and folding marker and an affinity tag.

44. The protein of claim 40, wherein said protein is the expression product of a Sh ble, Tn5 ble or Sa ble gene.

45. A method of immobilizing a protein to a surface, comprising providing the protein to the surface as a ble fusion protein and wherein the surface is a surface derivatized with an antibiotic from the bleomycin family.

46. The method of claim 45, wherein the antibiotic from the bleomycin family is selected from the group consisting of bleomycin, phleomycin, tallysomycin, pepleomycin and Zeocin™.

47. The method of claim 45, wherein the antibiotic from the bleomycin family is selected from the group consisting of bleomycin A2, bleomycin A5, bleomycin A6, bleomycin B2 or Zeocin™.

48. The method of claim 45, wherein a functional group on the antibiotic is used to link it to the surface.

49. The method of claim 48, wherein an amine group present on the antibiotic is used to couple the antibiotic to the surface.

50. The method of claim 49, wherein the antibiotic is coupled to a polyethyleneglycol (PEG) derivatized surface via an amine group.

51. The method of claim 45, wherein the surface is the surface of an array, a microtiter plate, a slide or a bead.

52. The method of claim 51, wherein the array is a microarray.

53. The method of claim 52, wherein the array is a MALDI array.

54. The method of claim 51, further comprising removing the ble fusion protein from the surface.

55. A probe comprising a target surface comprising an array having a plurality of discrete target areas presenting one or more analyte capture moieties comprising an antibiotic from the bleomycin family.

56. The probe of claim 55, wherein the antibiotic is provided on the target surface at a high surface density.

57. The probe of claim 56, wherein the capture moieties have an affinity for the moiety they are intended to capture in the order of 100 nM.

58. The probe of claim 55, wherein the antibiotic from the bleomycin family is selected from the group consisting of bleomycin, phleomycin, tallysomycin, pepleomycin and Zeocin™.

59. The probe of claim 55, wherein the antibiotic from the bleomycin family is selected from the group consisting of bleomycin A2, bleomycin A5, bleomycin A6, bleomycin B2 or Zeocin™.

60. A purification media comprising a large surface to volume area comprising a target surface presenting one or more analyte capture moieties comprising an antibiotic from the bleomycin family.

61. The purification media of claim 60 which is a bead.

62. The purification media of claim 60, wherein the antibiotic is provided on the target surface at a low surface density.

63. The purification media of claim 62, wherein the capture moieties have an affinity for the moiety they are intended to capture in the order of 600 nM.

64. The purification media of claim 60, wherein the antibiotic from the bleomycin family is selected from the group consisting of bleomycin, phleomycin, tallysomycin, pepleomycin and Zeocin™.

65. The purification media of claim 60, wherein the antibiotic from the bleomycin family is selected from the group consisting of bleomycin A2, bleomycin A5, bleomycin A6, bleomycin B2 or Zeocin™.

66. The purification media of claim 60, wherein the antibiotic is bound to the surface via a flexible linker molecule.

67. The purification media of claim 66, wherein the flexible linker molecule is a polyethylene glycol (PEG).

68. A method for generating soluble forms of an insoluble protein comprising the steps of:
i) generating a library of protein variants; and
ii) selecting colonies for the presence of a soluble protein by expressing the protein as a ble fusion protein and selecting an antibiotic from the bleomycin family.

69. The method of claim 68 further comprising the steps of growing the selected colonies, lysing them and binding the fusion protein to a surface.

70. The method of claim 69, wherein the surface comprises an antibiotic from the bleomycin family via which the fusion protein is bound.

71. A method of purifying a ble fusion protein from a crude extract comprising the step of immobilizing it on a surface via an antibiotic from the bleomycin family and optionally releasing it therefrom.

72. A method of identifying the cellular localization of a protein comprising the steps of:
i) expressing the protein as a ble fusion protein in a cell;
ii) introducing a labelled antibiotic from the bleomycin family into the cell; and
iii) detecting the labelled antibiotic.

73. The method of claim 72, wherein the antibiotic is an antibiotic from the bleomycin family characterized in that it is tagged with a marker.

74. The method of claim 73, wherein the marker is a visual marker.

75. The method of claim 74, wherein the visual marker is a fluorescent marker.

76. The method of claim 75, wherein the fluorescent marker is selected from NHS-activated fluoroscein, Cy3, Cy5, or Rhodamine.

77. A kit for the production of an array comprising a ble vector and a surface derivatized with an antibiotic from the bleomycin family or the components for making said derivatized surface.
